(19) United States
(12) Patent Application Publication
(10) Pub. No.: US 2004/0092583 A1
(43) Pub. Date: May 13, 2004

(54) Treatment for inhibiting neoplastic lesions

(76) Inventor: Elizabeth Shanahan-Prendergast, County Kildare (IE)

Correspondence Address: HOFFMANN & BARON, LLP, 6900 JERICHO TURNPIKE, SYOSSET, NY, 11791, US

(21) Appl. No.: 10/250,535

(22) PCT Filed: Jan. 2, 2002
(86) PCT No.: PCT/IE02/00001

(30) Foreign Application Priority Data
Jan. 2, 2001 (IE) S2001/0002

(51) Int. Cl. A61K 31/343; A61K 31/335; A61K 31/01

(52) U.S. Cl. 514/469; 514/475; 514/762

(57) ABSTRACT

The invention discloses the use of incensole and/or furanogermacrens, derivatives metabolites and precursors thereof in the treatment of neoplasia, particularly resistant neoplasia and immunodysregulatory disorders. These compounds can be administered alone or in combination with conventional chemotherapeutic, anti-rival, anti-parasite agents, radiation and/or surgery.

The present invention relates to a method for the selective inhibition of neoplastic cells, for example for the treatment, inhibition or prevention of precancerous lesions, tumours, cancer growth or other neoplasias in mammals. This invention also relates to the use of the compounds of the present invention including incensole and/or furanogermacren, derivatives, metabolites, analogues, mimic molecules and to compositions containing the compounds of the present invention including incensole and/or furanogermacren, derivatives, metabolites, analogues, mimic molecules.

Cancer develops from changes in the DNA, or genetic material, of the body's cells, causing them to develop into precancerous lesions. Such lesions exhibit a strong tendency to develop into malignant tumours, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms.

Cancer may take years to develop. The process typically begins with some disruption to the DNA of a cell, the genetic code that directs the life of the cell. Many things, such as diet, tobacco, sun exposure or certain chemicals can cause such disruptions. Some cells will enter a precancerous phase, known as dysplasia. Some cells will also enter the state of carcinoma in situ, in which the cancer cells are restricted to a microscopic site and do not pose a great threat. Eventually, unless the body's own immune system takes care of the wayward cells either on its own or by being enhanced by specific chemicals, a tumour will develop. It may take as long as 30 years for a tumour to go through the entire process and become large enough to produce clinical symptoms.

Anyone can get cancer, including children, but it is most common in people over the age of 50. This year about 1.22 million people in the United States will be diagnosed with cancer (not including the more than 1 million annual cases of basal and squamous-cell skin cancers.) About 563,000 people will die of cancer this year. Treatment for cancer has progressed rapidly over the last 30 years. Doctors generally prescribe three main treatments for cancer: surgery, radiation therapy, chemotherapy or a combination of these. Choosing a course of medical treatment depends largely on the cancer type, stage of progression, and location.

Surgery is generally advisable when physicians can safely remove the cancer from the body. In situations where the cancerous cells have spread, surgeons sometimes must remove large areas of healthy tissue along with the tumour to insure that no malignancy remains. In these cases, physicians remove lymph nodes from the tumour area because cancer can spread through nodes. However, unfortunately most cancers are discovered too late for surgical cure. In many cases, the patient does not experience symptoms until the cancer has progressed to a malignant stage.

Radiation therapy is used to destroy cancer cells. Ironically, radiation can cause and destroy cancer. Side effects of radiation therapy include radiation sickness, which are nausea and skin redness in the tumour area.

Chemotherapy uses poison drugs that take advantage of cancer cells' rapid growth and consumption of large amounts of nutrients. Chemotherapy side effects include nausea and temporary full or partial hair loss. Antimetabolites, one group of these drugs, work by mimicking the nutrients the body's cells consume. Physicians inject these drugs into the bloodstream, where they travel throughout the body, consumed by every cell. Rapidly growing cancerous cells consume much more of the poisonous drugs than do normal cells. As a result, the drugs destroy cancerous cells faster than normal cells. Cells reproduce by duplicating their genetic code, or DNA. Another group of chemotherapy drugs interferes with the duplication of DNA, so cells cannot reproduce. Chemotherapy drugs act on all the patients cells—the cancerous cells and the healthy cells. A physician's challenge is to administer the drugs to kill only the cancer cells, not the healthy cells. Side effects such as those immediately described prevent the long term or recurrent use of these drugs. Furthermore, there are an increasing number of effective drugs that can no longer be used due to resistance by the causative agent.

Researchers have refined these three cancer treatments (surgery, radiation therapy and chemotherapy) over the past 20 years. As a result, the survival rate among cancer patents has increased dramatically. But, the success of any treatment for cancer depends on how much the cancer has spread before treatment begins. Once cancer metastasises, or spreads into different areas of the body, treating it with surgery, radiation therapy or chemotherapy becomes more difficult. As the tumour mass increases and cancerous cells proliferate, the cancer may become resistant to any type of therapy medicine can provide.

Early cancer detection is critical to successful treatment. If physicians destroy tumours before they have had an opportunity to spread, a person with cancer has a much greater chance for survival.

The search for drugs useful for treating and preventing cancer is intensive. Indeed, much of the focus of cancer research today is on the prevention of cancer because chemotherapy for cancer itself is often not effective and has severe side effects. Cancer chemoprevention is important for recovered cancer patients whom retain a risk of cancer recurrence. Also, cancer chemoprevention is important for individuals who, have not yet had cancer, but have hereditary factors that place them at risk of developing cancer. With the development of new genetic screening technologies, it is easier to identify patients with high-risk genetic actors, whom would greatly benefit from chemopreventative drugs. Therefore finding such anti-cancer drugs that can be used for prolonged preventive use is of vital interest.

Unfortunately, most chemotherapeutic drugs have serious side effects that prohibit their long-term use, or use in otherwise healthy individuals with precancerous lesions. There side effects, which are a result of non-specific toxicity of the drugs, immunosuppression and other toxicities. For this reason there is a need to identify new drug candidates for therapy of patients with precancerous lesions that do not have such serious side effects in humans.

The in vitro anti-tumour activity of several natural products has recently been examined to identify new compounds that inhibit the cancer cells whilst having lower side effects, as described in U.S. Pat. No. 580,925; U.S. Pat. No. 6,051,565, U.S. Pat. No. 6,080,741; U.S. Pat. No. 5,578,637; U.S. Pat. No. 5,578,637; U.S. Pat. No. 5,663,196; U.S. Pat. No. 5,602,184; U.S. Pat. No. 5,817,816: and U.S. Pat. No. 56,077,840 to list but a few. A new group of drugs, utilizing monoclonal antibodies, designed to affect only cancer cells, leaving healthy cells intact have been tested. U.S. Pat. No. 5,064,823 discloses the anticancer activity of pentacyclic triterpenoid compounds which possess topoisomerase inhibitory activity. U.S. Pat. No. 5,876,728 is directed to a composition for treating cancer that contains at least three herbal extracts. These previously described compounds are unrelated to the present invention.

However, despite these developments, there exists a continuing need for chemotherapeutic agents which inhibit tumour growth, especially solid tumour growth and which have an adequate therapeutic index to be effective for in vivo treatment.

The correlation between the compounds of the present invention, incensole and furanogermacren, derivatives, metabolites, analogues and/or mimic molecules and neoplasia was not recognised prior to the work of the applicant. Accordingly the following provides information on each of these topics.

The present invention is directed to a composition comprising one or more compound of the present invention described herein, their derivatives, metabolites, analogues and/or mimic molecules with pharmaceutical acceptable additives, diluents, carriers, excipients and pharmaceutical salts thereof.

The present invention is further directed to a pharmaceutical formulation comprising a composition as described herein and pharmaceutically acceptable carrier thereof.

The present invention is further directed to the use of the pharmaceutical formulation for the manufacture of a medicament for treatment of a mammal suffering from a neoplasia comprising a pharmaceutical formulation as described herein.

The present invention is also directed to the use of the pharmaceutical formulation for the manufacture of a medicament for sensitising a resistant neoplasia to subsequent therapy comprising administering to a patient in need thereof a therapeutically effective amount of composition previously described.

The present invention is also directed to the use of a pharmaceutical formulation for the manufacture of a medicament for treatment of a mammal suffering from an immunodysregulatory condition comprising a composition as described herein to a subject.

### DETAILED DESCRIPTION OF THE INVENTION

The present invention is directed to a composition comprising one or more compound of the present invention described herein, their derivatives, metabolites, analogues and/or mimic molecules with pharmaceutical acceptable additives, diluents, carriers, excipients and pharmaceutical salts thereof.

In one embodiment, the compounds of the present invention are selected from the group comprising:

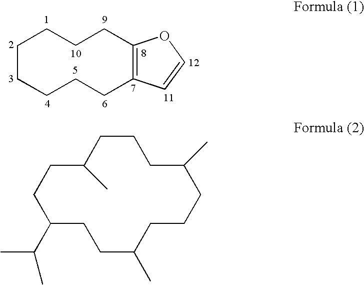

wherein for Formula (1)

Bonds between carbons 9-10, 10-1, 1-2, 2-3,3-4, 4-5, 5-6, can be either single or double with the proviso that any two or more double bonds are separated by a single bond.

Compounds also include those containing epoxide rings formed between carbons 9-10, 10-1, 1-2, 2-3,3-4, 4-5 with the proviso that any two or more epoxide rings are separated by a single bond.

wherein for Formula (2)

the carbocyclic ring can have optionally up to 7 endocyclic/exocyclic double bonds with the proviso that any two or more double bonds are separated by single bonds;

Carbon atoms for Formula (1) or (2) can be singly or multiply substituted, optionally and independently by:

an oxo substituent, H, alkyl, aryl, a heterocyclic radical, halogen, alkoxycarbonyl (C1-C5) or carboxyl, hydroxyl, alkoxy (C1-C5), amido, alkyl amido (C1-C5), amino, mono and dialkyl amino (C1-C5), alkyl carbamoyl (C1-C5), thiol, alkylthio (C1-C5);

in addition substituents may form a spiro ring around the carbon atom to which they are attached or they can form fused or bridged rings to adjacent carbon atoms which may be saturated or unsaturated;

Substituents on the aryl or heterocyclic radical are selected from the group consisting essentially of: halogen, alkyl (C1-C5), hydroxyl, alkoxy (C1-C5), alkoxycarbonyl, (C1-C5), carboxyl, imido, alkyl amido (C1 -C5), amino, mono and dialkyl amino (C1-C5),alkyl carbamoyl (C1-C5), thiol, alkyl thio (C1-C5) or benzenoid aryl thio, cyano, nitro, haloalkyl (C1-C5), alklsulfonyl (C1-C5), and sulfonate;

Two of such substituents can be part of a fused ring, which can be either saturated, or unsaturated, heterocyclic or carbo cyclic;

and natural amino acid substituents which may be attached to the compounds of formula (1) or (2) via an ester linkage to a hydroxyl group;

their derivatives, metabolites, analogues and/or mimic molecules with pharmaceutical acceptable additives, diluents, carriers and excipients and pharmaceutically acceptable salts thereof.

“Alkyl” as used herein means linked normal, secondary, tertiary or cyclic carbon atoms linear, branched or cyclic chains, saturated or unsaturated. The number of carbon atoms in an alkyl group or moiety is about 1 to about 20, unless otherwise specified, e.g. C1-10 alkyl means and alkyl moiety containing 1,2,3,4,5,6,7,8,9 or 10 carbon atoms. Substituents include but are not limited to halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5), alkyl carbamoyl (C1-C5), thiol, alkylthio (C1-C5) or benzenoid aryl.

“Aryl” as used herein refers to phenyl or naphthyl, or any optionally singly or multiply substituted benzenoid group (C6-C14). Substituents defined below.

“Heterocyclic” are used refers to any 4, 5 or 6 membered, optionally substituted heterocyclic ring, saturated or unsaturated, containing 1-3 ring heteroatoms, the remaining ring atoms being carbon.

In one embodiment, substituents on the aryl or heterocyclic radical may include hut are not limited to: halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5),alkyl carbamoyl (C1-C5), thiol, alkyl thio (C1-C5) or benzenoid aryl thio, cyano, nitro, haloalkyl (C1-C5), alklsulfonyl (C1-C5), and sulfonate.

Two of such substituents can be part of a fused ring, which can be either saturated, or unsaturated, heterocyclic or carbo cyclic.

In another embodiment, natural amino acid substituents which may be attached to the compounds of formula (1) or (2) via an ester linkage to a hydroxyl group.

In another embodiment, the compounds of the present invention are selected from the group comprising: incensole, incensole acetate, incensole oxide, incensole oxide acetate, isoincensole, isoincensole acetate, isoincensole oxide, octyl acetate, octanol, terpinyl acetate, bornyl acetate, trans-ver-benol, verbenone, menthadien-T7-ol, terpinen-4-ol, trans-pinocarveol, carvone, bomeol, farnesol, famesene, β-caryophyllene, humulene, β-cadinene, bergamotone, β-guaiene, β-ylangene, β-bourbonene, α-copaene, terpinene, myrcene, ρ-cymene, α- and β-phellandrene, α-thujene, cembrane-A, isocembrane, cambranol, cembranoids, cembranoid alcohols, furanogermacrens, furanogermacrene, gemacrene, elemane, cadinene, guaiane, oplopane, eudsmane, echinodol, α-santalene, α-bisabolene, furanodiene, β-santalene, β-bergamotene, β-famesene, β-bisabolene, SKB4, their derivatives, metabolites, analogues and/or mimic molecules with pharmaceutical acceptable additives, diluents, carriers and excipients and pharmaceutically acceptable salts thereof.

In another embodiment, the compounds of the present invention are selected from the group comprising at least one of: incensole, incensole acetate, incensole oxide, incensole oxide acetate, isoincensole, isoincensole acetate, isoincensole oxide, and/or at least one of the furanosesquiterpine furanogermacrens, their derivatives, metabolites, analogues and/or mimic molecules with pharmaceutical acceptable additives, diluents, carriers and excipients and pharmaceutically acceptable salts thereof.

In one embodiment, the composition is micronized. In accordance with the present invention, the expression “micronized” means that the composition has been micronized in accordance with any process for micronizing, a number of which are known in the art. The micronized particles preferably include a percentage of particles, which are of a diameter, which is about 10 microns, or less, preferably, 5 microns or less. For example, in a preferred aspect of the invention, at least 80% of the particles in a formulation of micronized particles have a diameter of less than 5 microns. An alternative to micronizing a compound is to solubilize the compound and put it into liposomes of appropriate size. The manufacture of liposomes and the insertion of active ingredients into such liposomes are well known in the art.

In another embodiment the composition is delivered to infected cells by incorporating the compounds of the present invention into liposomes or carbohydrate vehicles. In another embodiment, the composition is formulated into liposomes or carbohydrate vehicles.

In one embodiment, the liposomes or carbohydrate vehicles are specifically targeted to tumours by covalently attaching a monoclonal antibody directed to a tumour-associated antigen.

In one embodiment, the liposomes or carbohydrate vehicles are targeted to HIV infected cells by putting viral antibodies on its surface. In another embodiment, the viral antibodies are directed to the HIV coat protein gp160 and/or gp120.

In another embodiment, the present invention is directed to a pharmaceutical formulation comprising a composition as described herein and a pharmaceutically acceptable carrier thereof.

In one embodiment the pharmaceutically acceptable carrier, which in one embodiment is a cyclodextrin, alpha-cyclodextrin, beta-cyclodextrin, (beta-hydroxypropylcyclodextrin) gamma-cyclodextrin and in another embodiment is vitamin E oil.

The compounds of the present invention can be formulated and administered as free bases or in the form of their pharmaceutically acceptable salts for purposes of stability, convenience of crystallisation, increased solubility, and the like.

The present invention is further directed to the use of a pharmaceutical formulation for the manufacture of a medicament for treatment of a mammal suffering from a neoplasia comprising a pharmaceutical formulation as described herein.

The present invention is also directed to the use of a pharmaceutical formulation for the manufacture of a medicament for sensitising a resistant neoplasia to subsequent therapy comprising administering to a patient in need thereof a therapeutically effective amount of composition as previously described.

The present invention is directed to a method of inhibiting neoplastic cells by exposing those cells to a pharmacologically effective amount of compositions containing those compounds of the present invention described below, their derivatives, metabolites, analogues and/or mimic molecules with pharmaceutical acceptable additives, diluents, carriers and excipients. Such compounds are effective at eliminating and inhibiting the growth of neoplasias such as precancerous lesions, tumours and cancer growth. One of the advantages of utilising such compositions is that they are low in toxicity, which in combination with their mechanism of action diminishes resistance development.

The present invention provides compounds, their derivatives, metabolites, analogues and/or mimic molecules with pharmaceutical acceptable additives, diluents, carriers and excipients and pharmaceutically acceptable salts thereof, as well as pharmaceutical compositions comprising the compounds of the present invention, their derivatives, metabolites, analogues and/or mimic molecules with pharmaceutical acceptable additives, diluents, carriers and excipients and methods comprising inhibiting tumour growth or treating cancer by administering one or more of the compounds of the present invention, their derivatives, metabolites, analogues and/or mimic molecules with pharmaceutical acceptable additives, diluents, carriers and excipients.

The present invention also provides products that are useful for treating neoplasia with minimal toxic side effects unlike the high toxicity associated with standard chemotherapeutic agents.

The present invention is also directed to the use of a pharmaceutical formulation for the manufacture of a medicament for treatment of a mammal suffering from an immunodysregulatory condition comprising a composition as described herein to a subject.

The present invention is also directed to providing a composition that regulates immune responses.

These and other objects of the present invention will become apparent from the description of the invention disclosed below, which descriptions are intended to limit neither the spirit or scope of the invention but are only offered as illustrations of the preferred embodiments of the invention.

The present invention is directed to the treatment, inhibition and/or prevention of tumours and/or cancer growth and more particularly to treating neoplasia.

As used herein, the term “neoplasia” or neoplasm covers dysplasia, precancerous lesions, cancerous lesions, neoplastic cells, cancer, cancer growth, tumours, benign tumours, malignant tumours, solid tumours, carcinomas, etc.

As used herein, the term “precancerous lesion” includes syndromes represented by abnormal neoplastic, including dysplastic, changes of tissue. Examples include precancerous growths in colonic, breast, renal, central nervous, gastric, or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not

The compounds of the present invention can be administered to a mammal having a susceptible cancer, i.e. a malignant cell population or tumour. Compounds of the present invention are effective on human tumours in vivo as well as on tumour cell lines in vitro. The compounds of the present invention may be particularly useful for the treatment of solid tumours for which relatively few treatments are available. Such tumours include epidermoid and myeloid tumours, acute or chronic, nonsmall cell, squamous. Specific cancers which may be mentioned as susceptible to treatment by administration of compounds in accordance with the present invention include prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system (based on the likelihood that the compounds will cross the blood cell barrier) including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas. Hematopoeitic cell cancers such as B cell leukaemia/lymphomas, myelomas, T-cell leukemias/lymphomas, small cell leukemias/lymphomas, null cell, sezary, monocytic, myelomonocytic and Hairy cell leukemias. These lymphomas/leukemias can be either acute or chronic. Other cancers may also be susceptible to treatment with the compounds of the present invention. The activity can readily be measured using standardised tests known to those skilled in the art.

As used herein, the term “carcinomas” refers to lesions that are cancerous. Examples include malignant melanomas, breast cancer, and colon cancer. As used herein, the term “neoplasm” refers to both precancerous and cancerous lesions.

As used herein, the terms “inhibit” or “inhibiting,” mean decreasing tumour cell growth rate from the rate that would occur without treatment and/or causing tumour mass to decrease. Inhibiting also includes causing a complete regression of the tumour. Thus the compounds of the present invention can be either cytostatic or cytotoxic to the tumour cells.

As used herein, the terms subject and patent are used interchangeably. Subjects and patients are mammals.

The compounds of the present invention are useful antineoplastic agents i.e. to inhibit tumour cell growth in vitro and in vivo, in mammalian hosts, such as humans or domestic animals, and are particularly effective against solid tumours and multidrug resistant tumours. Thus the invention provides a method comprising inhibiting cancer cells, by contacting said cells, in vitro and in vivo with an effective amount of at least one compound of the present invention. The invention also provides a therapeutic method comprising treating cancer (i.e. inhibiting tumour cell growth) by administering at least one of the compounds of the present invention to a mammal in need of such therapy.

The invention is directed to a method of treating tumours comprising administering a biologically active amount of a composition which consists of at least one compound of the present invention, their derivatives, metabolites, analogues and/or mimic molecules with pharmaceutical acceptable additives, diluents, carriers and excipients and pharmaceutically acceptable salts thereof.

The invention features a method of treating cancer comprising administering to a patient in need thereof a cancer treatment effective amount of a composition which consists of at least one compound of the present invention, their derivatives, metabolites, analogues and/or mimic molecules with pharmaceutical acceptable additives, diluents, carriers and excipients and pharmaceutically acceptable salts thereof.

It was surprisingly found that when a composition which consists of at least one compound of the present invention or their derivatives, metabolites, analogues or mimic molecules were administered, the proliferation of neoplastic cells was inhibited, which is manifested, pursuant to one aspect of the present invention, in a broad-spectrum anti-neoplastic activity.

The compounds of the present invention are individually diverse, but collectively all act to inhibit the propagation of neoplastic cells, cancers, cancer growth and/or tumours.

The invention also features a method of treating neoplasia comprising administering to a patient an effective amount of a composition which consists of at least one compound of the present invention and a pharmaceutically acceptable carrier.

Treating neoplasia in a patient includes achieving, partially or substantially, one or more of the following: arresting the growth or spread of a cancer, reducing the extent of a cancer (e.g., reducing size of a tumour or reducing the number of affected sites). Inhibiting the growth rate of a cancer, and ameliorating or improving a clinical symptom or indicator associated with a cancer (such as tissue or serum components).

The present invention relates to specific compounds, defined herein, that display immuno-modulatory activity. It has been surprisingly found the compounds of the present invention have potent immuno-modulatory activity.

The present invention also relates to compositions and methods of treatment for prevention of an immunodysregulation condition.

The present invention also relates to specific compounds, defined herein, that enhance endogenous heat shock proteins (hsp) and precursor dendritic cell levels.

In accordance with the present invention, a method is provided to treat or prevent an immunodysregulatory condition comprising administering to a subject an effective amount of a composition which consists of at least one of the compounds of the present invention, their derivatives, metabolites, analogues and/or mimic molecules with pharmaceutical acceptable additives, diluents, carriers and excipients and pharmaceutically acceptable salts thereof.

The present invention also provides the use of compositions which consist of one or more of the compounds of the present invention, their derivatives, metabolites, analogues and/or mimic molecules with pharmaceutical acceptable additives, diluents, carriers and excipients and pharmaceutically acceptable salts thereof, for the manufacture of a medicament for an immunodysregulatory condition.

The present invention also provides compositions which consists of at least one compound of the present invention for use in a method of treatment of an immunodysregulatory condition, said method comprising administering one or more to a subject.

The pharmaceutical formulations may also be administered in combination with other therapeutic treatments, such as radiation treatment, surgery or in combination with other anticancer, antiviral or antiparasite drugs. The formulations of the present invention may further include as optional ingredients one or more chemotherapeutic agents already known for their use in the inhibition of cancer cells for added clinical efficacy. Such combinations will in some cases provide added benefit.

In one embodiment, the pharmaceutical formulation further includes at least one conventional chemotherapeutic agent.

In another embodiment, the conventional chemotherapeutic agent is selected from the group comprising flutamide and luprolide, antioestrogens, such as tamoxifen, antimetabolites and cytotoxic agents, such as daunorubicin, flourouracil, floxuridine, interferon alpha, methotrexate, plicamycin, mecaptopurine, thinguanine, adramycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cispletin, streptozocin, bleomycin, dactinomycin and idamycin, hormones such as, medroxyprogesterone, estramustine, ethinyl oestradiol, oestradiol, leuprolide, megestrol, octreotide, diethylstilbestrol, chlorotrianisene, etoposide, podophyllotoxin, and goserelin, nitrogen mustard derivatives such as, melphalan, chlorambucil, methlorethamine and thiotepa, steroids such as, betamethasone, and other antineoplastic agents such as live Mycobacterium bovis, dicarbazine, asparaginase, leucovoribn, mitotane, vincristine, vinblastine and texotere, cyclophosphamide, adriamycin, 5-flourouracil, hexamethylmelamine, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthremycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Carecemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate, Duazomycin; Edatrexate; Eflomithine Hydrochloride; Elsamitrucin; Enloplatin; Enpromate; Epipropidine; Epirubicin Hydrochloride, Erbulozole; Esorubicin Hydrochloride Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine, Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine: Mechlorethamine Hydrochloride, Megestrol Acetate: Melengestrol Acetate; Melphalan; Menoageril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin: Ormaplatin; Oxisuran; Paclitaxel; Pegaspargase; Pellomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safrngol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Tallsomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; arnifostine; aminolevulinic acid; amrubicin; atrsacrine; anagrelide; anastrozole; andrographolide; angiogenesis inhibitors; antagonist D; antagonist G; DHEA; bromineepiandrosterone; epiandrosterone; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2: axinastatin 3; azasetron; azatoxin: azatyrosine; baccatin III derivatives; balanol; batimastat; BCR/ABL antagonists; benzochlorins; benzoylslaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin R4; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; torfenimex; formestane; fostriecin; fotemustine; gadolinium texaphyrin; gallium nitrate; galocitabine; ganirelix; gelatinase inhibitors; gemcitabine; glutathione inhibitors, hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor, interferon agonists; interferons; interleukins; iobenguane; lododoxarubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat: masoprocol; maspin; matrilysin inhibitors; matrix metalloproteinase inhibitors menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant, nitrullyn; O6-benzylguanine; octreotide; okicenone; oligonucteotides; onapristone; ondansetron; ondansetron; oracin; oral cytokine inducer, ornaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds: platinum-triamine complex; porfimer sodium; porfiromycin; propyl bis-acridone, prostaglandin J2; proteasome inhibitors; protein A-based immune modulator; protein kinase C inhibitor; protein kinase C inhibitors, microalgal; protein tyrosine-phosphatase inhibitors; purine nucleoside phosphorylase inhibitors, purpurins; pyrazoloacridine; pyridoxylated hemoglobin polyoxyethylon conjugate; raf entagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators, single chain antigen binding protein; sizofiran; sobuzoxane; sodium borocaptate; sodium phenylacetate; solverol; sornatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine, superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin: toremifene: totipotent stem cell factor, translation inhibitors; tretinoin; triacetyluridine; triciribine; trimetrexate; triptorelin; tropisetron, turosteride, tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy, velaresol; venom, anti-venom, veramine; verdins; verteporfin; vinorelbine; vinxaltine: vitaxin; vorozole, zanoterone; zeniplatin; zilascorb; zinostatin stimalarner, immunostimulating drugs or therapeutic agents, their metabolites, salts and derivatives thereof.

In one embodiment, the composition further includes at least one anti-viral agent.

In one embodiment, the anti-viral agents are selected from the group comprising nucleoside analogues (AZT; ddC; ddl; d4T; 3TC; BW 1592; PMEA/bis-POM PMEA; dOTC; DAPD); non-nucleoside reverse transcriptase inhibitors (delavirdine; DMP 266; HBY097; loviride; nevirapine, emivirine; AG1549; PNU142721, Calanolide A; DPC961), protease inhibitors (ABT-378; ritonavir, nelfinavir; BW 141; KNI-272; indinavir, saquinavir; L-756,423; DMP-450; BMS-232630); ALX40-4C; hydroxyurea; lobucavir, pentafuside; T-1249; PRO 542; FP-21399; AMD 3100; HE-2000 and peptide T; Abacavir: Acemannan; Acyclovir; Acyclovir Sodium; Adefovir; Alovudine; Alvircept Sudotox; Amantadine Hydrochloride; Aranotin; Arildone; Atevirdine Mesylate; Avricline; Cidofovir; Cipamfylline; Coviracil; Cytarabine Hydrochloride; Delavirdine Mesylate; Desciclovir, Didanosine; Disoxaril; Edoxudine; Emivirine; Emtricitabine; Enviradene; Enviroxime; Epivir; Famciclovir, Famotine Hydrochloride; Fiacitabine; Fialuridine; Fosarilate; Foscarnet Sodium; Fosfonet Sodium; Ganciclovir; Ganciclovir Sodium; Idoxuridine; Indinavir; Kethoxal; Lamivudine; Lobucavir, Lodenosine; Lopinavir, Memotine Hydrochloride; Methisazone: Nelfinavir; Nevirapine; Penciclovir, Pirodavir; Ribavirin; Rimantadine Hydrochloride; Saquinavir Mesylate; Ritonavir; Somantadine Hydrochloride; Sorivudine; Statolon; Stavudine; Tenofovir; Tilorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine; Zinviroxime and Intereron.

In one embodiment, the composition further includes at least one anti-parasite agent.

In one embodiment, the anti-parasite agents are selected from the group comprising chloroquin, primaquine, mefloquine, pyrimethamine-sulfadoxone, atoraquone/dapsone; halofantrine; artemisinin derivatives; atoraquone+proguanol, co-artemether, podophyllotoxin; pentamidine, ditoxanide furoate, metronidazole, tindazole, tetracycline, quinacrine, stibogluconate, amphotericin B, quinine, doxycline, trimethoprim-sulfamethoxazole, metronidazole, nifurtimox, suramin, melarsoprol, benznidezole, their metabolites, salts derivatives or any other anti-parasitic agent thereof.

The agents of the present invention may also be administered in combination with other agents for example immunostimulating drugs or therapeutic agents. Appropriate amounts in each case will vary with the particular agent, and will be either readily known to those skilled in the art or readily determinable by routine experimentation.

In one embodiment, the pharmaceutical formulation is administered in combination with radiation treatment.

In one embodiment, the pharmaceutical formulation is administered in combination with surgery.

The invention also relates to a method of suppressing tumour growth in a mammal by administering to the mammal an amount of a composition which consist of at least one of the compounds of the present invention, derivatives, metabolites, analogues and/or mimic molecules, and a second chemotherapeutic agent effective to suppress tumour growth in the mammal. The second chemotherapeutic agent is not a compound of the present invention or a derivative, metabolite, analogue or mimic molecule. These compositions provide enhanced antitumour effect and may also prevent the development of metastases. In particular, these compounds are useful for overcoming tumours that are drug resistant. These agents may be administered separately or as a cocktail. Toxicity may be reduced by administering the compound of the present invention or a derivative, metabolite, analogue or mimic molecule, thereof several hours prior to administering the chemotherapeutic agent. The compositions can be administered by any route.

The components of any of the pharmaceutical formulations disclosed herein can be administered simultaneously (in a combination formulation), essentially simultaneously (e.g., administration of each compound a few minutes or a few hours apart), or can be administered sequentially, e.g., several days apart, or more than a week apart. For example, a compound of the present invention, (and a conventional chemotherapeutic agent) can be administered together, or essentially simultaneously, e.g., administration of each compound a few minutes or a few hours apart, or can be administered sequentially, e.g., several days apart, or more than a week apart. All such variations in administration of the combination therapy are encompassed within the scope of the invention.

In one embodiment, the neoplasia is a precancerous lesion including syndromes represented by abnormal neoplastic and/or dysplastic, changes of tissue comprising precancerous growths in colonic, breast, renal, central nervous, gastric, or lung tissues, or conditions such as dysplastic nevus syndrome, a precursor to malignant melanoma of tho skin, dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic karatosis), whether the lesions are clinically identifiable or not.

In one embodiment, the neoplasia is prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas. Hematopoeitic cell cancers such as B cell leukaemia/lymphomas, myelomas, T-cell leukemias/lymphomas, small cell leukemias/lymphomas, null cell, sezary, monocytic, myelomonocytic and Hairy cell leukemias.

In one embodiment, the neoplasia is in the form of a tumour comprising an epidermoid and myeloid tumour, acute or chronic, nonsmall cell, squamous or solid.

It has been surprisingly found that the compounds of the present invention have potent immuno modulatory effects, Accordingly, the disclosed compounds of the present invention when administered to human patients will have a broad immuno-modulatory effect, resulting in its application in many syndromes, especially following treatment for or infection by cancerous cells. More specifically, one aspect of the present invention relates to the use of the compounds of the present invention in treatment of an immunodysregulation condition.

According to one aspect of the invention, a composition containing at least one of the compounds of the present invention enhance the production of endogenous heat shock protein (hsp) production, regardless of the immunocompetency of the individual. According to another aspect of the invention, a composition containing at least one of the compounds of the present invention enhance levels of precursor dendritic cells.

The advantage of administering a pharmaceutical formulation containing at least one compound of the present invention with a suitable carrier is three fold:

1. The cancer-infected cell is presented to the immune system where its presence is detected due to enhanced immunosurveillence.

2. The compounds of the present invention have immuno up-regulatory properties, precursor dendritic and natural killer cells are up regulated. Antigen capture is further enhanced by a domino effect of increasing precursor dendritic cell maturation into cytotoxic T cells.

3. The presentation of the antigenic peptides to the cytotoxic T cells is improved.

In one embodiment, a pharmaceutical formulation is provided to enhance endogenous hsp levels, comprising administering to a patient in need thereof a therapeutically effective amount of a composition comprising at least one compound of the present invention.

In another embodiment, a method for enhancing endogenous hsp levels in a living subject is provided, comprising administering to a patient in need thereof a therapeutically effective amount of a composition comprising at least one compound of the present invention.

In another embodiment, a method of treating the symptoms of low levels of endogenous hsp levels in a living subject is provided, comprising administering to a patient in need thereof a therapeutically effective amount of a composition comprising at least one compound of the present invention.

In another embodiment, a pharmaceutical formulation is provided to enhance endogenous precursor dentritic cell levels, comprising administering to a patient in need thereof a therapeutically effective amount of a composition comprising at least one compound of the present invention.

Additionally, the invention provides use of the composition to provide protection against infections in immunocompromised animals and humans. These compositions may be used prophylactically or therapeutically to protect animals or patients from the consequences of infection by pathogens.

Further, the invention provide use of tho composition in veterinary medicine, prophylactically and therapeutically in animal populations that are subject to infection that compromises immune response and cause infection.

In another aspect of the invention, the use of a pharmaceutical formulation is provided for the manufacture of a medicament for the treatment of a mammal suffering from an immuno dysregulation condition, comprising administering to a patient in need thereof a therapeutically effective amount of a a pharmaceutical formulation of the present invention.

In one embodiment, the immuno dysregulation condition is caused by a viral infection, intracellular bacterial infection, extracellular bacterial infection, fungal infection, yeast infection, extracellular parasite infection, intracellular parasite infection, protozoan parasite, multicellular parasite, autoimmune disease, immunosuppressive therapy, chemotherapy, anti-infective agent therapy, wound, burn, the presence of an immunosuppressive molecule, gastrointestinal irritation or any combination of the foregoing and is selected from (a) a DNA virus infection or an RNA virus(b) a parasite infection, a Trypanosoma, Plasmodium, Cryptosporidium, Entamoeba, Balangidium, Leishmania, Pneumocystis, Trichomoniasis or Toxoplasma infection, wherein the Trypanosoma, Plasmodium, Cryptosporidium, Entamoeba, Balatidium, Leishmania; Pneumocystis, Trichomoniesis or Toxoplasma infection is selected: from but not limited to Trypanosoma cruzi, Trypanosoma brucei, Trypanosoma gambiense, Trypanosoma rhodesiense, Plasmodium falciparum, Plasmodium vivax, Plasmodium malariae, Plasmodium ovale, Plasmodium berghei, Entamoeba histolytica, Balantidium coli, Leishmania brazilienis, Leishmania mexicana, Leishmania donovani, Leishmania tropica, Pneumocystis carinii, Trichomoniasis vaginalis, and Toxoplasma gondii (c) a mycoplasma infection, a Listeria infection or a Mycobacterium infection; (d) a Streptococcus infection, a Staphylococcus infection, a Vibrio infection, a Salmonella infection; a Shigella infection, an enterotoxigenic, enteropathogenic, enteroinvasive or enterohemorrhagic E. coli infection, a Yersinia infection, a Campylobacter infection, a Pseudomonas infection, a Borrelia infection, a Legionella infection and a Haemophilus infection; (e) pulmonary Aspergillosis, mucosal or oropharyngealcandidlasis and juvenile paracoccidiomyosis; (f) a Candida infection and a Cryptococcus infection; (g) systemic lupus erythematosis, arthritis, asthma, and diabetes (h) adriamycin treatment, cisplatin treatment, mitomycin C treatment, amphoteracin B treatment; (i) a gamma-radiation is treatment; (j) nucleoside analog treatment for viral infection or for cancer; (k) surgical and accidental wounds, septic shock caused by surgery; (l) cyclosporin treatment and corticosteroid treatment; (m) irritable bowel treatment, Crohn's disease, wasting syndrome, cachexia, Motor Neuron disease, Multiple Sclerosis, inflammatory bowel disease, respiratory distress syndrome, chronic diarrhoea; (n) cancer; (o) cirrhoisis; (p) gram positive multi-drug resistant bacteria or (q) any combination of (a) through (p).

In one embodiment, the DNA virus infection or the RNA virus infection is selected from a retrovirus infection, a togavirus infection, a flavivirus infection, a rubivirus infection, a pestivirus infection, a lipid envelope virus infection, a filovirus, a picornavirus infection, a rhinovirus infection, a coronavirus infection, a respiratory synoytial virus infection, a poliovirus infection, a parainfluenza virus infection, influenza virus infection, hantavirus, adeno-associated virus, measles virus, poxvirus, filovirus, human papilloma virus and animal papilloma virus infection.

In one embodiment, the composition enhances endogenous hsp levels.

In one embodiment, the composition enhances endogenous precursor dendritic cell levels.

The invention also relates to a method for reducing the immunodepressive effect of a chemotherapy agent in a mammal by administering to a mammal an amount of a composition which consists of at least one compound of the present invention or a derivative, metabolite, analogue or mimic molecule thereof effective to augment the immune system of the mammal upon treatment of the mammal with the chemotherapeutic agent. The immune system may be augmented, for example, by increasing the total number of leukocytes, T-lymphocytes, B-lymphocytes, or immunoglobulins.

The present invention also provides a method of sentisizing a neoplasia to subsequent treatment, for example radiation or chemotherapy. It is known that cancer cells become resistant to some chemotherapeutic agents and are even resistant to many different chemotherapeutic agents. This is a significant to many different chemotherapeutic treatments. This method includes administering an effective amount of a composition which consists of at least one compound of the present invention or a derivative, metabolite, analogue or mimic molecule thereof to a mammal having cancer.

In another aspect of the invention, the use of a pharmaceutical formulation is provided for the manufacture of a medicament for sensitising a resistant neoplasia to subsequent therapy comprising administering to a patient in need thereof a therapeutically effective amount of a a pharmaceutical formulation of the present invention.

The method of the present invention is useful in sensitizing desensitized cancer cells in particular: ovarian, sarcoma, non-Hodgkin's lymphoma, lung, breast cancer, bladder carcinoma, colon carcinoma, pancreatic carcinoma, carcinoma of the ampulla of Vater, multiple myeloma, adult acute lymphocytic leukemia, adult non-lymphoytic leukemia and neuroblastoma. It is preferred that the cancer cells be breast cancer, multiple myeloma, ovarian or lung.

It is realized that the desensitized cancer cells may be desensitized to more than one chemotherapeutic agent. If so, the method of the present invention will sensitize the desensitized cancer cells to most of the chemotherapeutic agents to which they are desensitized.

In one embodiment, the chemotherapeutic agents to which the cancer cells become desensitized are selected from the group consisting of doxorubicin, daunomycin, vinca alkaloids, vincristine, vinblastine, taxol, colchicine, epipodophyllotoxins such as etoposide, actinomycin D, puromycin, emetine, melphalan, adozalesin, [S-(R,R)]6,6′-[carbonylbis(imino-1H-indole-05,2-diylcarbonyl)]bis[8-(chloromethyl) 3,6,7,8-tetrahydro-1-methyl-benzo[1,2-b;4,3-b′]dipyrrol-4I, (S)-N-[2-[[1-(chloromethyl)-1,6-dihydro-3-methyl-5-[[(phenylamino)carbonyl]opxy]benzo[1,2-b:4,3-b′]dipyrrol-3(2H)-yl]carbonyl]-1H-indol-5-yl]-6-(diethylamino)-2 benzofurancarboxamide, (7bR, 8aS)-7-[[1,6-dihydro-4-hydroxy-5-methoxy-7-[(4,5, 8, 8a-tetrahydro-7-methyl-4-oxocyclopropa[c]pyrrolo[3,2-e]indol-2(1H)-yl)carbonyl]benzo[1,2-b:4,3-b′]dipyrrol-3(2H)-yl]carbonyl]-1,6-dihydro-4-hydroxy-5-methoxybenzo[1,2-b:4,3-b′]dipyrrole-3(2H)-carboxamide.

It is realized that new chemotherapeutic agents against cancer will be developed after this invention. The new chemotherapeutic agents to which resistance develops and which can be treated by the method of this invention are equivalent to those set forth in this invention.

In one embodiment, the pharmaceutical formulation has an enteric coating. In one embodiment, the enteric coating is made of a polymer or copolymer. In one embodiment, the polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

The pharmaceutical formulation according to the present invention can be administered to a patient in any of a wide range of routes. Thus, with regard to the types of formulations in which the active compounds according to the present invention can be administered, as well as any additives can be included with the active compounds in the formulations, and the possible routes of administration, it is well known to those of skill in the art that such formulations can be provided in a wide variety of types, and it is within the skill of the ordinary artisans to select a specific formulation and route of administration and then test suitability for use. By way of example but not limitation, suitable routes include enteric, parenteral, topical, oral, rectal, nasal or vaginal routes. Parenteral routes include subcutaneous, intramuscular, intravenous, intraperitoneal, intradermal and subilingual administration. Also, compositions may be implanted into a patient or injected using a drug delivery system.

The pharmaceutical formulation according to the present invention may be administered locally or systemically. By systemic administration means any mode or route of administration that results in effective amounts of active ingredient appearing in the blood or at a site remote from the route of administration of the active ingredient.

Further, the pharmaceutical formulation according to the present invention may be administered intermittently. The advantage of this is that it allows the patient to suspend therapy for periods without the worry of inactivity of the drug resulting from the development of resistant cells.

The pharmaceutical formulation according to tho invention may be formulated for enteral, parenteral or topical administration. Indeed all three types of formulations may be used simultaneously to achieve systemic administration of the active ingredient.

Compounds useful in the methods of this invention may be formulated into compositions together with pharmaceutically acceptable carriers for oral administration in solid or liquid form, or for rectal administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S. Additional pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavouring and perfuming agents.

Pharmaceutically acceptable carriers for rectal administration are preferably suppositories that may contain, in addition to the compounds of the present invention, excipients such as cocoa butter or a suppository wax.

Suitable injectable solutions include intravenous, subcutaneous and intramuscular injectable solutions. Examples of injectable forms include solutions, suspensions and emulsions. Typically the compound(s) is injected in association with a pharmaceutical carrier such as normal saline, Ringers solution, dextrose solution and other aqueous carriers known in the art. Appropriate non-aqueous carriers may also be used and examples include cyclodextrin, preferably hydroxypropyl beta cyclodextrin, mixed oils (vitamin E oil), polyethylene glycol and ethyl oleate. A preferred carrier is cyclodextrin in water. Frequently, it is desirable to include additives in the carrier such as buffers and preservatives or other substances to enhance isotonicity and chemical stability.

The composition can also be administered topically. Suitable formulations for topical administration include creams, gels, jellies, mucliages, pastes and ointments. The compounds may be formulated for transdermal administration, for example in the form of transdermal patches so as to achieve systemic administration.

The composition may also be administered in the form of an implant.

The composition may also be administered in the form of an infusion solution or as a nasal inhalation, aerosol or spray.

In another embodiment, the composition is incorporated in a pharmaceutically acceptable carrier, diluents, vehicles and the like for systemic administration by feeding. An example of such a carrier is cyclodextrin (α-cyclodextrin, β-hydroxypropylcyclodextrin or γ-cyclodextrin).

In one embodiment, the pharmaceutical formulation is administered enterally, parenterally, topically, orally, sub-lingually, rectally, nasally or vaginally.

In one embodiment, the pharmaceutical formulation is administered to a mammal. In one embodiment, said mammal is a neonate and said administering is effected prior to delivery of said neonate and/or during delivery of said neonate.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, individual needs and other factors. If desired, the unit dosage may be such that the daily requirement for active compound is in one dose, or divided among multiple doses for administration, e.g., two to four times per day.

With regard to dosage and duration of treatment according to any aspect of the present invention, it is recognized that the ability of an artisan skilled in pharmaceutical administration of drugs to determine suitable dosages depending on many inter-related factors is well known, and skilled artisans are readily able to monitor patients to determine whether treatment should be started, continued, discontinued or resumed at any given time. For example, dosages of the compounds are suitably determined depending on the individual cases taking symptoms, age and sex of the subject and the like into consideration. The amount of the compound to be incorporated into the pharmaceutical composition of the invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum is effect is achieved. Suitable dosages can be determined by further taking into account relevant disclosure in the known art. In one embodiment, the unit dose comprises 5-500 mg of active ingredient consisting of at least one compound of the present invention.

The pharmaceutical formulations of this invention are preferably packaged in a container (e.g. a box or bottle, or both) with suitable printed material (e.g. a package insert) containing indications, directions for use, etc.

The present invention is also directed to compositions which consist of at least one compound if the present invention acting as prodrug compounds analogous to the active compounds disclosed herein. Such compounds are generally themselves inactive or low in activity, but are converted into active compounds. Thus, for example, pro-drugs such as the methyl ester of any acid functionality, which is not active per se or has very low activity could be hydrolysed, either uncatalytically or catalytically with an enzyme such as an esterase to an active compound. Such pro-drug compounds could well be the preferred therapeutic form of the present compounds. These analogous prodrugs can be produced from active compounds based on procedures and factors that are well known to one of ordinary skill in the art. Accordingly as used in the present application, “pro-drug analogue” means “a chemical which is relatively non-toxic and pharmacologically inert but which can be transformed in vivo to a pharmacologically active drug”. More specifically it means a derivative, metabolite or analogue of the compounds of the present invention which have low or no ability as anti-neoplastic agents until converted in the body to a derivative, metabolite or analogue with such ability or abilities. Such pro-drugs should have favourable properties such as enhanced absorption, water solubility, lower toxicity, or better targeting to the tumour cell (such as by reason of greater affinity to the tumour cell or a larger quantity of activating enzyme in the tumour cell as opposed to a normal cell so that larger concentrations of the active compound are produced in the tumour cell). Examples of such compounds are esters, such as methyl, ethyl, phenyl, N,N-dimethylaminoethyl, acyl derivatives such as benzoyl, p-N,N-dimethylaminobenzoyl, N,N-dimethylaminoglycyl, peptide derivatives such as γ-glutamyl, glycyl, D-Val-Leu-Lys.

In one embodiment, said compounds of the present invention acts as a prodrug.

The compositions containing the active compounds or pro-drugs of the present invention can be formulated so as to be specifically targeted to tumours. The compounds can be attached to the reagent that is capable of binding a tumour-associated antigen. For example, the compounds of the present invention could be covalently attached to a monoclonal antibody such as directed to a tumour-associated antigen. The antigen may be located on a tumour or in the tumour cell area. Such linkages can be made through peptide bond formation with amino groups of an antibody. Suitable reagents include polyclonal and monoclonal antibodies. Accordingly, the present invention also provides a method comprising treating cancer (i.e. inhibiting tumour cell growth) by administering a pharmaceutical composition comprising at least one of the compounds of the present invention and a reagent (i.e. monoclonal or polyclonal antibody) which is capable of binding to a tumour-associated antigen.

Alternatively, the compounds of the present invention could be attached to or incorporated into liposomes or carbohydrate vehicles, which are known to be useful far targeting anti-cancer drugs. Preferably the liposomes or carbohydrate vehicles can be specifically targeted to tumours by covalently attaching a monoclonal antibody directed to a tumour-associated antigen.

The invention further provides a composition for treating a cancer selected from the group consisting of small cell lung cancer, testicular cancer, lymphoma, leukaemia. oesophageal cancer, stomach cancer, colon cancer, breast cancer, central nervous system cancer, liver cancer and prostate cancer, which comprising administering to a mammal in need thereof an effective amount of a composition containing as an active ingredient therein at least one of the compounds of the present invention, their derivatives, metabolites, analogues and/or mimic molecules with pharmaceutical acceptable additives, diluents, carriers, excipients and pharmaceutical salts thereof.

The invention provides a method for inducing cellular differentiation, which comprises contacting a cancerous cell with an effective amount of at least one compound of the present invention or a derivative, metabolite, analogue or mimic molecule and pharmaceutically acceptable salts thereof.

The invention provides pharmaceutical formulations which consist of compositions comprising, at least one compound of the present invention and corresponding pharmaceutically acceptable derivatives, metabolites, analogues, mimic molecules and mixtures thereof are to be used as anti-neoplasic and/or anti-cancer agents.

The invention provides pharmaceutical formulations to be used in the preparation of medicaments having anti-neoplastic and/or anti-cancer activity.

The invention further provides, the use of the pharmaceutical formulations as anti-neoplasic and/or anticancer agents.

In another embodiment, the use of pharmaceutical formulations for the preparation of medicaments having anti-neoplastic and/or anti-cancer activity.

In another embodiment, pharmaceutical formulations are provided, for the preparation of medicaments having activity against neoplasm and/or cancer.

The present invention is exemplified in terms of in vitro and in vivo activity against various neoplastic cell lines. The test cell lines employed in the in vitro assays are well recognised and accepted as models for anti-tumour activity in animals. The term animals as used herein includes, but is not limited to, mice, rats, domesticated animals such as but is not limited to, cats, dogs, and other animals but is not limited to, cattle, sheep, pigs, horses, and primates such as but not limited to, monkeys, humans and more generally mammals.

Without further elaboration, it is believed that one skilled in the art can, using the preceding description, practice the present invention to its fullest extent. The following detailed examples describe how to test the various compounds of this invention and/or perform the various processes of the invention and are to be construed as merely illustrative, and not limitations of the preceding disclosure in any way whatsoever. Those skilled in the art will promptly recognize appropriate variations from the procedures.

The active components with anti-neoplastic activity were extracted from resins of plants from the plant family Burseraceae, primarily Myrrh (Commiphora spp.) and Frankincense or Olibanum (Boswellia carteri). The components were extracted from the resins using standard extraction techniques followed by chromatographic isolation. Sample components were identified using several methods including preparative HPLC, mass spectroscopy, NMR spectroscopy, and IR and UV spectroscopy.

Multiple components can be extracted from the resins including: incensole, incensole acetate, incensole oxide, incensole oxide acetete, isoincensole, isoincensole acetate, isoincensole oxide, octyl acetate, octanol, terpinyl acetate, bornyl acetate, trans-ver-benol, verbenone, menthadien-7-ol, terpinen-4-ol, trans-pinocarveol, carvone, borneol, farnesol, farnesene, β-caryophyllene, humulene, β-cadinene, bergamotone, β-guaiene, β-ylangene, β-bourbonene, α-copaene, terpinene, myrcene, ρ-cymene, α- and β-phellandrene, α-thujene, cembranes, cembrane-A, isocembrane, cembranol, cembranoids, cembranoid alcohols, furanogermacrens, furanogermacrene, germacrene, elemane, cadinene, guaiane, oplopane, eudsmane, echinodol, α-santalene, α-bisabolene, furanodiene, β-santalene, β-bergamotene, β-farnesene, β-bisabolene, T-cadinol, SKB4.

It is thought that multiple components could contribute to the anti-neoplasic components of the extracts. However the most active anti-neoplasic components comprised the diterpenoids incensole, incensole acetate, incensole oxide, incensole oxide acetate, isoincensole, isoincensole acetate, isoincensole oxide, and the furanosesquiterpene fumogermecrens in highest concentrations.

### EXAMPLE 1

In vitro cytotoxic activity of extracts containing high concentrations of incensole, furanogermacren and incensole/furanogermacren mixture were determined in several cultured tumour cell lines by performing the MTT clonogenic assay. This assay assesses the inhibition of colony formation of tumour stem cells growing in soft agar by cytotoxic agents. Since tumour stem cells are responsible for the proliferate potential and aggressiveness of a tumour cell population, the clonogenic assay is highly predictive of in vivo response.

### Drugs and Chemicals

The extracts were dissolved in DMSO/water 1:1, prepared as a 30 mg/ml solution and stored at −20° C. until use. Final dilutions of all drugs were prepared in culture medium immediately prior to use. 5-Flourouracil was used as a positive control and purchased from Lederle (Hamburg).

### Clonogenic Assay

### Single Cell Suspension From Tumour Cell Lines

Human tumour cell lines were grown in RPMI 1640 supplemented with 10% fetal calf serum and 2 mM L-glutamine. The cells were kept at 5% CO₂ and 37° C. and passaged routinely. For treatment experiments, exponentially growing cells were trypsinized, washed twice with PBS and the percentage of viable cells was determined by hemocytometer count using trypan blue viable dye exclusion.

### Culture Methods

The clonogenic assay was performed according to a modified two-layered soft agar assay. The bottom layer consisted of 0.2 mls of Iscove's Modified Dulbecco's Medium with 20% fetal calf serum and 0.75% agar. 2.5×104 cells in RPMI/10% FCS were added in 0.2 ml medium, but containing 0.4% agar and placed in 24-multiwell plates on top of the base layer. After 24 hours, drugs were added in additional 0.2 ml of RPMI medium. Every plate contained 6 vehicle controls and 6 different drug concentrations in triplicate. 5-flourouracil was used as a positive control in concentrations of 100, 300 and 1000 μg/ml. Cultures were incubated at 5% CO₂ and 37° C. in a humidifled atmosphere for 5-6 days under continuous exposure to drugs and monitored closely for colony growth using an inverted microscope. Within this period, in vitro tumour growth led to the formation of colonies with a diameter of 50 μm. At the time of colony formation, counts were performed with automated image analysis system (OMNICOM FAS IV, Biosys GmbH). Vital colonies were stained with a sterile aqueous solution of 2-(4-iodophenyl)-3-(4nitrophenyl)-5-phenyltetrazolium chloride (1 mg/ml, 100 μl/well) 24 hours prior to evaluation. Drug effect was assessed in terms of percentage of survival obtained by comparison to the mean number of colonies in the treated wells with mean colony count of the untreated controls: treated controls×100 (% T/C).

A compound was considered active if it reduces the colony formation to 30% or less of the control group value (T/C 30%). IC₅₀ and IC₇₀ values, representing the drug concentration to inhibit colony formation by 50% (T/C 50%) and 70% (T/C 30%) respectively, were determined by plotting compound concentration versus T/C values. Mean IC₅₀ and IC₇₀ values were calculated according to the following formula:

$$\frac{\begin{matrix}3 log ( {IC}_{70} ) x \\ X = 1\end{matrix}}{N}$$

Mean IC₇₀=10

x=specific tumour cell line

n=total number of cell lines studied

If an ID₅₀ or IC₇₀ value could not be determined within the examined dose range, the lowest or highest concentration studies was used for the calculation. An assay was considered valuable if the following criteria were fulfilled:

1. Mean number of colonies in the control group dishes for 24-multiwells contained 20 colonies with colony diameters >50 μm.

2. The positive reference 5-flourouracil (at toxic dose of 1000 μg/ml) must affect colony survival of 30% of controls.

Coefficient of variation in the control group <50%>

The extracts were tested in several tumour cell lines, for their ability to affect tumour growth. The cell lines tested included: HT29 colon carcinoma; SF 268 central nervous system; GXF 251L gastric cancer, LXFE 66NL epidermoid lung carcinoma; LXFL 529L large cell lung carcinoma; H460 lung adenocarcinoma; LXFF6 529L lung adenocarcinoma; MCF-7 breast cancer; OVCAR3 ovarian carcinoma: PG3 prostate carcinoma; DU145 prostate carcinoma; RXF 944L renal call carcinoma; MEXF 514L melanoma; and MEXF 426NL melanoma.

The IC50 results are for in vitro studies are presented in Table 1 below:

| | | | | IC₅₀ μm/ml | |
|---|---|---|---|---|---|
| | Code | | | Incensole/ | IC₅₀ μm/ml |
| | Cancer Cell | IC₅₀ μm/ml | IC₅₀ μm/ml | Furanogermacren | 5- |
| Cancer Cell Line | Line | Incensole | Furanogermacren | Mixture | Flourouracil |
| Human Prostate | DU145 | ND | ND | 5.00 | 0.021 |
| Carcinoma | | | | | |
| Human Prostate | PC3 | 6.00 | 70.00 | 1.2 | ND |
| Carcinoma | | | | | |
| Human Colon | HT29 | 70.00 | 70.00 | 0.9 | ND |
| Carcinoma | | | | | |
| Human Melanoma | MEXF 514L | ND | ND | 0.8 | ND |
| Human Melanoma | MEXF | ND | ND | 20.00 | ND |
| | 429NL | | | | |
| Human Lung | LXFE 66NL | ND | ND | 50.00 | ND |
| Epidermoid Carcinoma | | | | | |
| Human Lung Large | LXFL 529L | ND | ND | 4.00 | ND |
| Cell Carcinoma | | | | | |
| Human Lung | H460 | ND | ND | 20.00 | ND |
| Adenocarcinoma | | | | | |
| Human Renal | RXF 944L | ND | ND | 30.00 | ND |
| Carcinoma | | | | | |
| Human Breast | MACL | ND | ND | 7.00 | ND |
| Carcinoma | MCF7 | | | | |
| Gastric Carcinoma | GXF 251L | ND | ND | 8.00 | ND |
| Ovarian Cancer | OVCL | ND | ND | 10.00 | ND |
| Xenograft | OVCAR3 | | | | |
| Carcinoma of the | CNCL | ND | ND | 10.00 | ND |
| central nervous system | SF288 | | | | |

ND = not determined

### EXAMPLE 2

### Antimicrobial Activity

These studies were performed to determine the minimum inhibitory concentration (MIC) of incensole/furanogermacren mixture.

MIC values were determined using a macrodiluton broth procedure based on NCCLS Documents M7-A3 “Methods for dilution anti-microbial susceptibility tests for bacteria that grow aerobically-third edition, approved standard (1993). The lowest test substance concentration that completely inhibited growth of the test organisms recorded ac MIC.

The following organisms were tested:

| | |
|---|---|
| 1. | Staphylococcus aureus NCTC 10442 |
| 2. | Staphylococcus aureus NCTC 6571 |
| 3. | Enterococcus feacalis NCTC 775 |

MIC values for incensole/furanogermacren mixture were 100 μg/ml. MIC values for incensole for the above 3 bacterial species was 38 μg/ml causing significant growth retardation.

Pharmaceutically acceptable refers to those properties and/or substances, which are acceptable to the patient from a pharmacological/toxicological point of view including bioavailability and patient acceptance or to the manufacturing chemist from a physical-chemical point of view regarding composition, formulation, stability and isolatability.

The terms “comprise, comprised and comprising” and the terms “include, included and including” are used interchangeably in this specification and are to be afforded the widest interpretation.

The invention is not limited to the embodiments described above, but may be varied in both construction and detail within the scope of the claims.

We claim

1. A composition comprising one or more compound of the present invention, their derivatives, metabolites, analogues and/or mimic molecules with pharmaceutical acceptable additives, diluents, carriers, excipients and pharmaceutical salts thereof.

2. The composition as claimed in claim 1, wherein the compounds of the present invention are selected from the group comprising: 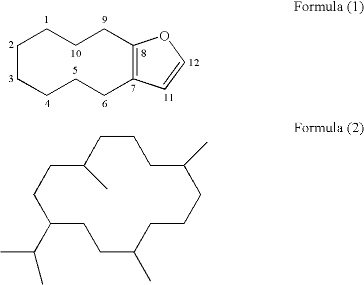
wherein for formula (1)
bonds between carbons 9-10, 10-1, 1-2, 2-3,3-4, 4-5, 5-6, can be either single or double with the proviso that any two or more double bonds are separated by a single bond.
Compounds also include those containing epoxide rings formed between carbons 9-10, 10-1, 1-2, 2-3,3-4, 4-5 with the proviso that any two or more epoxide rings are separated by a single bond.
wherein for Formula (2)
the carbocyclic ring can have optionally up to 7 endocyclic/exocyclic double bonds with the proviso that any two or more double bonds are separated by single bonds;
Carbon atoms for Formula (1) or (2) can be singly or multiply substituted, optionally and independently by:
an oxo substituent, H, alkyl, aryl, a heterocyclic radical, halogen, alkoxycarbonyl (C1-C5) or carboxyl, hydroxyl, alkoxy (C1-C5), amido, alkyl amido (C1-C5), amino, mono and dialkyl amino (C1-C5), alkyl carbamoyl (C1-C5), thiol, alkylthio (C1-C5);
in addition substituents may form a spiro ring around the carbon atom to which they are attached or they can form fused or bridged rings to adjacent carbon atoms which may be saturated or unsaturated;
Substituents on the aryl or heterocyclic radical are selected from the group consisting essentially of: halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5),alkyl carbamoyl (C1-C5), thiol, alkyl thin (C1-C5) or benzenoid aryl thio, cyano, nitro, haloalkyl (C1-C5), alklsulfonyl (C1-C5), and sulfonate;
Two of such substituents can be part of a fused ring, which can be either saturated, or unsaturated, heterocyclic or carbo cyclic;
and natural amino acid substituents which may be attached to the compounds of formula (1) or (2) via an ester linkage to a hydroxyl group;
their derivatives, metabolites, analogues and/or mimic molecules with pharmaceutical acceptable additives, diluents, carriers and excipient and pharmaceutically acceptable salts thereof.

3. The composition as claimed in claims 1 or 2, wherein the compounds of the present invention are selected from the group comprising: incensole, incensole acetate, incensole oxide, incensole oxide acetate, isoincensole, isoincensole acetate, isoincensole oxide, octyl acetate, octanol, terpinyl acetate, bornyl acetate, trans-ver-benol, verbenone, menthadien-7-ol, terpinen-4-ol, trans-pinocarveol, carvone, borneol, farnesol, farnesene, β-caryophyllene, humulene, β-cadinene, bergamotone, β-guaiene, β-ylangene, β-bourbonene, α-copaene, terpinene, myrcene, ρ-cyrnene, α- and β-phellandrene, α-thujene, cembrane-A, isocembrane, cembranol, cembranoids, cembranoid alcohols, furanogermacrens, furanogermacrene, germacrene, elemane, cadinene, guaiane, oplopane, eudsmane, echinodol, α-santalene, α-bisabolene, furanodiene, β-santalene, β-bergamotene, β-farnesene, β-bisabolene, SKB4, their derivatives, metabolites, analogues and/or mimic molecules with pharmaceutical acceptable additives, diluents, carriers and excipients and pharmaceutically acceptable salts thereof.

4. The composition as claimed in any preceding claims, wherein the compounds of the present invention are selected from the group comprising at least one of: incensole, incensole acetate, incensole oxide, incensole oxide acetate, isoincensole, isoincensole acetate, isoincensole oxide, and/or at least one of the furanosesquiterpene fumogermacrens, their derivatives, metabolites, analogues and/or mimic molecules with pharmaceutical acceptable additives, diluents, carriers and excipients and pharmaceutically acceptable salts thereof.

5. A pharmaceutical formulation comprising a composition as claimed in any of claims 1 to 4 and a pharmaceutically acceptable carrier thereof.

6. The pharmaceutical formulation as claimed in claim 5, wherein the pharmaceutical carrier is selected from the group comprising cyclodextrin, alpha-cyclodextrin, beta-cyclodextrin, (beta-hydroxypropylcyclodextrin) gamma-cyclodextrin and vitamin E oil.

7. The use of a pharmaceutical formulation for the manufacture of a medicament for treatment of a mammal suffering from a neoplasia comprising a pharmaceutical formulation as claimed in claims 1 to 6.

8. The use of a pharmaceutical formulation as claimed in any of the preceding claims, wherein the pharmaceutical formulation further includes at least one conventional chemotherapeutic agent.

9. The use of a pharmaceutical formulation as claimed in claim 7, wherein the chemotherapeutic agent is selected from the group comprising flutamide and luprolide, antioestrogens, such as tamoxifen, antimetabolites and cytotoxic agents, such as daunorubicin, flourouracil, floxuridine, interferon alpha, methotrexate, plicamycin, mecaptopurine, thioguanine, adramycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozooin, bleomycin, dactinomycin and idamycin, hormones such as, medroxyprogesterone, estramustine, ethinyl oestradiol, oestradiol, leuprolide, megestrol, octreotide, diethylstilbestrol, chlorotrianisene, etoposide, podophyllotoxin, and goserelin, nitrogen mustard derivatives such as, melphalan, chlorambucil, methlorethamine and thiotepa, steroids such as, betamethazone, and other antineoplastic agents such as live Mycobacterium bovis, dicarbazine, asparaginase, leucovoribn, mitotane, vincristine, vinblastine and texotere, cyclophosphamide, adriamycin, 5-flourouracil, hexamethylmelamine, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azptepa: Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribino; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin, Edatrexate; Eflomithine Hydrochloride; Elsamitrucin; Enloplatin; Enpromate; Epipropidine, Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide: Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole: Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safrngol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate: Trimetrexate; Trimetrexate Glucuronate; Triptorelin; Tubulozole Hydrochloride; Uracil Mustard; Urodepa; Vapreotide; Verleporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine: amidox; amifostine: aminolevulinic acid; amrubicin; atrsacrine; anagrelide; anastrozole; andrographolide; angiogenesis inhibitors; antagonist D; antagonist G; DHEA; bromineepiandrosterone; epiandrosterone; antarelix; anti-dorselizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capedtabine; carboxamide-amino-triazole: carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrmzoxane; dexverapamil; diaziquorie; didemnin 13; didox; diethylnorspermine; dihydro-5-azacytidine: dihydrotaxol, 9-; dioxamycin; diphenyl spiromustine; docosanol; dolasetron; doxifluridine; droloxifene; dronabinol; duocannycin SA; ebselen; ecorustine; edelfosine; edrocolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; fligrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors: gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides: insulin-like growth factor-1 receptor inhibitor; interferon agonists; interferons; interleukins; iobenguane; iododoxorubicin; ipomeanol, 4-; irinolecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetlum texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin: monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline: N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone: ondansetron; ondansetron; oracin; oral cytokine inducer; ormaplatin; osaterone; oxaliplatin; oxaunomycin; paclitaxel analogues; paclitaxel derivatives; palauamine; palmitoylrizoxin; pamidronic acid; panaxytriol; panomifene; parabactin; pazelliptine: pegaspargase; peldesine: pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; propyl bis-acridone, prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors: ras-GAP inhibitor; retelliptine demethylated; rhenium Re 186 etidronate; rhizoxin; ribozymes; RII retinamide; rogletimide; rohitumine; romurtide; roquinimex; rubiginone B1; ruboxyl; safingol; saintopin: SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; splcamycin D; spiromustine: splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur, tellurapyrylium; telomerase inhibitors; telnoporfin; ternozolomide; teniposide; tetrachlorodecaoxide; tetrazomine; thallblastine; thalidomide; thiocoraline; thrombopoietin; thrombopoietin mimetic; thymalfasin; thymopoietin receptor agonist; thymotrinan; thyroid stimulating hormone; tin ethyl otiopurpurin; tirapazamine; titanocene dichloride; topotecan; topsentin; toremifene; totipotent stem cell factor; translation inhibitors; tretinoin; triacetyluridine; triciribine; trimetrexate; triptorelin: tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; venom, anti-venom, veramine; verdins; verteporfin; vinorelbine; vinxaltine; vitaxin; vorozole; zanoterone; zeniplatin; zilascorb; zinostatin stimalamer, immunostimulating drugs or therapeutic agents, their metabolites, salts and derivatives thereof.

10. The use of a pharmaceutical formulation as claimed in claims 7 to 9, wherein the pharmaceutical formulation is administered in combination with radiation treatment.

11. The use of a pharmaceutical formulation as claimed in claims 7 to 10, wherein the pharmaceutical formulation is administered in combination with surgery.

12. The use of a pharmaceutical formulation as claimed in claims 7 to 11, wherein the neoplasia is a precancerous lesion including syndromes represented by abnormal neoplastic and/or dysplastic, changes of tissue comprising precancerous growths in colonic, breast, renal, central nervous, gastric, or lung tissues, or conditions such as dysplastic nevus syndrome, a precursor to malignant melanoma of the skin, dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

13. The use of a pharmaceutical formulation as claimed in claims 7 to 12, wherein the neoplasia is prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas, Hematopoeitic cell cancers such as B cell leukaemia/lymphomas, myelomas, T-cell leukemias/lymphomas, small cell leukemias/lymphomas, null cell, sezary, monocytic, myelomonocytic and Hairy cell leukemias.

14. The use of a pharmaceutical formulation as claimed in claims 7 to 13, wherein the neoplasia is in the form of a tumour comprising an epidermoid and myeloid tumour, acute or chronic, nonsmall cell, squamous or solid.

15. The use of a pharmaceutical formulation as claimed in claims 7 to 14, wherein the composition is micronized.

16. The use of a pharmaceutical formulation as claimed in claims 7 to 15, wherein the pharmaceutical formulation has an enteric coating.

17. The use of a pharmaceutical formulation as claimed in claim 16, wherein the enteric coating is made of a polymer or copolymer.

18. The use of a pharmaceutical formulation as claimed in claim 17, wherein the polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

19. The use of a pharmaceutical formulation as claimed in claims 7 to 18, wherein, the pharmaceutical formulation is administered enterally, parenterally, topically, orally, sub-lingually, rectally, nasally or vaginally.

20. The use of a pharmaceutical formulation as claimed in claims 7 to 19, wherein the composition is formulated into liposomes or carbohydrate vehicles.

21. The use of a pharmaceutical formulation as claimed in claim 20, wherein the liposomes or carbohydrate vehicles are specifically targeted to tumours by covalently attaching a monoclonal antibody directed to a tumour-associated antigen.

22. The use of a pharmaceutical formulation as claimed in claims 7 to 21, wherein the pharmaceutical formulation is administered intermittently.

23. The use of a pharmaceutical formulation as claimed in claims 7 to 22, wherein the pharmaceutical formulation is a unit dose that comprises 5-500 mg of active ingredient consisting of at least one compound of the present invention.

24. The use of a pharmaceutical formulation as claimed in claims 7 to 23, wherein the pharmaceutical formulation is administered to a mammal.

25. The use of a pharmaceutical formulation as claimed in claim 24, wherein said mammal is a neonate and said administering is effected prior to delivery of said neonate and/or during delivery of said neonate.

26. The use of a pharmaceutical formulation as claimed in claims 7 to 25, wherein said compounds of the present invention acts as a prodrug.

27. The use of a pharmaceutical formulation for the manufacture of a medicament for treatment of a mammal suffering from an immunodysregulatory condition comprising a composition as claimed in claims 1 to 6, to a subject.

28. The use of a pharmaceutical formulation as claimed in claim 27, wherein the immunodysregulation condition is caused by a viral infection, intracellular bacterial infection, extracellular bacterial infection, fungal infection, yeast infection, extracellular parasite infection, intracellular parasite infection, protozoan parasite, multicellular parasite, autoimmune disease, immunosuppressive therapy, chemotherapy, anti-infective agent therapy, wound, burn, the presence of an immunosuppressive molecule, gastrointestinal irritation or any combination of the foregoing is selected from (a) a DNA virus infection or an RNA virus(b) a parasite infection, a Trypanosoma, Plasmodium, Cryptosporidium, Entamoeba, Balantidium, Leishmania, Pneumocystis, Trichomoniasis or Toxoplasma Infection, wherein the Trypanosoma, Plasmodium, Cryptosporidium, Entamoeba, Balantidium, Leishmania, Pneumocystis, Trichomoniasis or Toxoplasma infection is selected from but not limited to Tryponosoma cruzi, Tryponosoma brucei, Trypanosoma gambiense, Trypanosoma rhodesiense, Plasmodium falciparum; Plasmodium vivax, Plasmodium malariae, Plasmodium ovale; Plasmodium berghei, Entamoeba histolytica, Balantidium coli, Leishmania brazillenis, Leishmania mexicana, Leishmania donovani, Leishmania tropica, Pneumocystis carinii, Trichomoniasis vaginalis, and Toxoplasma gondii (c) a mycoplasma infection, a Listeria infection or a Mycobacterium infection; (d) a Streptococcus infection, a Staphylococcus infection, a Vibrio infection, a Salmonella infection; a Shigella infection, an enterotoxigenic, enteropathogenic, enteroinvasive or enterohemorrhagic E. coli infection, a Yersinia infection, a Campylobacter infection, a Pseudomonas infection, a Borrelia infection, a Legionella infection and a Haemophilus infection; (e) pulmonary Aspergillosis, mucosal or oropharyngealcandidiasis and juvenile paracoccidiomyosis, (f) a Candida infection and a Cryptococcus infection; (g) systemic lupus erythematosis, arthritis, asthma, and diabetes (h) adriamycin treatment, cisplatin treatment, mitomycin C treatment, amphoteracin B treatment; (i) a gamma-radiation treatment; (j) nucleoside analog treatment for viral infection or for cancer, (k) surgical and accidental wounds, septic shock caused by surgery; (l) cyclosporin treatment and corticosteroid treatment; (m) irritable bowel treatment, Crohn's disease, wasting syndrome, cachexia, Motor Neuron disease, Multiple Sclerosis, inflammatory bowel disease, respiratory distress syndrome, chronic diarrhoea; (n) cancer, (o) cirrhoisis; (p) gram positive multi-drug resistant bacteria or (q) any combination of (a) through (p).

29. The use of a pharmaceutical formulation as claimed in claim 28, wherein the DNA virus infection or the RNA virus infection is selected from a retrovirus infection, a togavirus infection, a flavivirus infection, a rubivirus infection, a pestivirus infection, a lipia envelope virus infection, a filovirus, a picornavirus infection, a rhinovirus infection, a coronavirus infection, a respiratory syncytial virus infection, a poliovirus infection, a parainfluenza virus infection, influenza virus infection, hantavirus, adeno-associated virus, measles virus, poxvirus, filovirus, human papilloma virus and animal papilloma virus infection.

30. The use of a pharmaceutical formulation as claimed in claims 27 to 29, wherein the composition further includes at least one conventional chemotherapeutic agent.

31. The pharmaceutical formulation as claimed in claim 30, wherein the chemotherapeutic agent is selected from the group comprising flutamide and luprolide, antioestrogens, such as tamoxifen, antimetabolites and cytotoxic agents, such as daunorubicin, flourouracil, floxuridine, interferon alpha, methotrexate, plicamycin, mecaptopurine, thioguanine, adramycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin and idamycin, hormones such as, medroxyprogesterone, estramustine, ethinyl oestradiol, oestradiol, leuprolide, megestrol, octreotide, diethylstilbestrol, chlorotrianisene, etoposide, podophyllotoxin, and goserelin, nitrogen mustard derivatives such as, melphalan, chlorambucil, methlorethamine and thiotepa, steroids such as, betamethasone, and other antineoplastic agents such as live Mycobacterium bovis, dicarbazine, asparaginase, laucovoribn, mitotone, vincristine, vinblastine and texotere, cyclophosphamide, adriamycin, 5-flourouracil, hexamethylmelamine, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunonibicin Hydrochloride; Decitabine; Dexormaplatin; Dezaguanine; Dezaguanine Mesylate; Diaziquone; Docetaxol; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine: Fludirabine Phosphate; Fluouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide: Mitocarcin; Mitocromin; Mitogillin; Mitomalcin; Mitomycin; Mitosper; Mitotane; Mitoxantrone Hydrochloride; Mycophenolic Acid; Nocodozole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safrngol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Tallsomycin; Taxane; Taxoid; Tecogalan Sodium; Tegafur; Teloxantrone Hydrochloride; Temoporfin; Teniposide; Teroxirone; Testolactone: Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorein; Tubulozole Hydrochloride; Uracil Mustard; Uredepa, Vapreotide; Verteporfin; Vinblastine Sulfate; Vincristine Sulfate; Vindesine; Vindesine Sulfate; Vinapidine Sulfate; Vinglycinate Sulfate; Vinieurosine Sulfate; Vinorelbine Tartrate; Vinrosidine Sulfate; Vinzolidine Sulfate; Vorozole; Zeniplatin; Zinostatin; Zorubicin Hydrochloride, 20-epi-1,25 dihydroxyvitamin D3; 5-ethynyluracil; abiraterone; aclarubicin; acylfulvene; adecypenol; adozesin; aldesleukin; ALL-TK antagonists; altretemine; ambamustine; amidox; amifostine; aminolevulinic add; amrubicin; atrsacrine; anagrelide; anastrazole; andrographoilde; angiogenesis inhibitors; antagonist D; antagonist G; DHEA, bromineepiandrosterone; epiandrosterone; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid: ara-CDP-DL-PTSA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine: beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C: camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophydn A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; doxifosfamide; dexrazoxane; dexverapamil; diaziquone; didemnin B; didox; diethylnorspermine; dihydro-5-azacytidine; dihydrotaxol, 9; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur, epirubicin; epristeride; estramustine analogue; estrogen agonists; estrogen antagonists; etanidazole; etoposide phosphate; exemestane; fadrozole; fazarabine; fenretinide; filgrastim; frnasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors: gemcitabine; glutathione inhibitors; hepsulfam; heregulin; hexamethylene bisacetamide; hypericin; ibandronic acid; idarubicin; idruxifene; idramantone; ilmofosine; ilomastat; imidazoacridones; imiquimod; immunostimulant peptides; insulin-like growth factor-1 receptor inhibitor; interferon agonists; interferons; interleukins; iobenquane; iododoxorubicin; ipomeanol, 1-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine: lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF Inhibitor; mirfepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonedotrophin; monophosphoryl lipid A+myobacterium cell wall sk; mopidamol; multiple drug resistance genie inhibitor; multiple tumorsuppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron, ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perlilyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein-based immune modulator; protein kinase C inhibitor; protein kinase C inhibitors, microalgal; protein tyrosine phosphitase inhibitor; purin nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors: ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin: tropisetron; turosteride; tyrosine kinase inhibitors; tyrphostins; UBC inhibitors; ubenimex; urogenal sinus-derived growth inhibitory factor; urokinase receptor antagonists; vapreotide; variolin B; vector system, erythrocyte gene therapy; velaresol: venom, anti-venom, veramine; verdins; verteporfin; vinorelbine; vinxaltine; vitaxin; vorozole; zanoterone; zeniplatin; zilascorb; zinostatin stimalamer, immunostimulating drugs or therapeutic agents, their metabolites, salts and derivatives thereof.

32. The use of a pharmaceutical formulation as claimed in claims 27 to 31, wherein the composition further includes at least one anti-viral agent.

33. The use of a pharmaceutical formulation as claimed in claim 32, wherein the anti-viral agents are selected from the group comprising nucleoside analogues (AZT; ddC; ddl; d4T; 3TC; BW 1592; PMEA/bis-POM PMEA; dOTC; DAPD); non-nucleoside reverse transcriptase inhibitor (delavirdine; DMP 266; HBY097; loviride; nevirapine, emivirine; AG1549; PNU142721; Calanolide A; DPC961); protease inhibitors (ABT-378; ritonavir; nelfinavir; BW 141; KNI-272; indinavir; saquinavir; L-756,423; DMP-450: BMS-2326;30); ALX40-4C; hydroxyurea; lobucavir; pentafuside; T-1249; PRO 542: FP-21399; AMD 3100; HE-2000 and pepude T; Abacavir, Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Coviracil; Cytarabine Hydrochloride; Delavirdine Mesylate; Desciclovir, Didanosine; Disoxaril; Edoxudine; Emivirine; Emtricitabine; Enviradene; Enviroxime; Epivir; Famciclovir; Famotine Hydrochloride; Fiacitabine; Fialuridine; Fosarilate; Foscarnet Sodium: Fosfonet Sodium; Ganciclovir, Ganciclovir Sodium; Idoxuridine; Indinavir; Kethoxal; Lamivudine; Lobucavir; Lodenosine; Lopinavir, Memotine Hydrochloride; Methisazone; Nelfinavir; Nevirapine; Penciclovir; Pirodavir; Ribavirin; Rimantadine Hydrochloride; Saquinavir Mesylate; Ritonavir; Somantadine Hydrochloride; Sorivudine; Statolon; Stavudine; Tenofovir; Tilorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine; Zinviroxime and Interferon.

34. The use of a pharmaceutical formulation as claimed in claims 27 to 33, wherein the composition further includes at least one anti-parasite agent.

35. The use of a pharmaceutical formulation as claimed in claim 34, wherein the anti-parasite agents are selected from the group comprising chloroquin, primaquine, mefloquine, pyrimethamine-sulfadoxone, atoraquone/dapsone; halofantrine; artemisinin derivatives; atoraquone+proguanol, co-artemether; podophyllotoxin; pentamidine, diloxanide furoate; metronidazole, tindazole, tetracycline, quinacrine, stibogluconate, amphotericin B, quinine, doxycline, trimethoprim-sulfamethoxazole, metronidazole, nifurtimox, suramin, melarsoprol, benznidazole, their metabolites, salts derivatives or any other anti-parasitic agent thereof.

36. The use of a pharmaceutical formulation as claimed in claims 27 to 35, wherein the composition is micronized.

37. The use of a pharmaceutical formulation as claimed in claims 27 to 36, wherein the composition enhances endogenous hsp levels.

38. The use of a pharmaceutical formulation as claimed in claims 27 to 37, wherein the composition enhances endogenous precursor dendritic cell levels.

39. The use of a pharmaceutical formulation as claimed in claims 27 to 38, wherein the pharmaceutical formulation has an enteric coating.

40. The use of a pharmaceutical formulation as claimed in claim 39, wherein the enteric coating is made of a polymer or copolymer.

41. The use of a pharmaceutical formulation as claimed in claim 40 wherein the polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

42. The use of a pharmaceutical formulation as claimed in claims 27 to 41, wherein the pharmaceutical formulation is administered enterally, parenterally, topically, orally, rectally, nasally or vaginally.

43. The use of a pharmaceutical formulation as claimed in claims 27 to 42, wherein the composition is formulated into liposomes or carbohydrate vehicles.

44. The use of a pharmaceutical formulation as claimed in claim 43, wherein the liposomes or carbohydrate vehicles are targeted to HIV infected cells by putting viral antibodies on its surface.

45. The use of a pharmaceutical formulation as claimed in claim 44, wherein the viral antibodies are directed to the HIV coat protein gp160 and/or gp120.

46. The use of a pharmaceutical formulation as claimed in claims 27 to 45, wherein the pharmaceutical formulation is administered intermittently.

47. The use of a pharmaceutical formulation as claimed in claims 27 to 46, wherein the pharmaceutical formulation is administered to a mammal.

48. The use of a pharmaceutical formulation as claimed in claim 47, wherein said mammal is a neonate and said administering is effected prior to delivery of said neonate and/or during delivery of said neonate.

49. The use of a pharmaceutical formulation as claimed in claims 27 to 48, wherein said compounds of the present invention acts as a prodrug.

50. The use of a pharmaceutical formulation for the manufacture of a medicament for sensitising a resistant neoplasia to subsequent therapy comprising administering to a patient in need thereof a therapeutically effective amount of composition as claimed in claims 1 to 6.

51. The use of a pharmaceutical formulation as claimed in claim 50, wherein the pharmaceutical formulation further includes at least one conventional chemotherapeutic agent.

52. The use of a pharmaceutical formulation as claimed in claim 51, wherein the chemotherapeutic agent is selected from the group comprising flutamide and luprolide, antiestrogens, such as tamoxifen; antimetabolites and cytotoxic agents, such as daunorubicin, flourouracil, floxuridine, interferon alpha, methotrexate, plicamycin, mecaptopurine, thioguanine, adramycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin and idamycin, hormones such as, medroxyprogesterone, estramustine, ethinyl oestradiol, oestradiol, leuprolide, megestrol, octreotide, diethylstilbestrol, chlorotrianisene, etoposide, podophyllotoxin, and goserelin, nitrogen mustard derivatives such as, melphalan, chlorambucil, methlorethamine and thiotepa, steroids such as, betamethasone, and other antineoplastic agents such as live Mycobacterium bovis, dicarbazine, asparaginase, leucovoribn, mitotane, vincristine, vinblastine and texotere, cydophosphamide, adriamycin, 5-flourouracil, hexamethylmelamine, Acivicin; Aclarubicin; Acodazole Hydrochloride; AcrQnine; Adozolesin; Aldesloukin; Altretamine; Ambomycin; Ametantrone Acetate; Aminogluthimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomyrin; Edatrexate; Eflomithine Hydrochloride; Elsamitrucin; Enloplatin; Enprorfate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane: Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride: Pyrazofurin: Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirono; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, 20-epi-1,25 dihydroxyvitamin D3; 5-ethynyluracil; abiraterone, aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; DHEA; bromineepiandrosterone; epiandrosterone; antarelix; anti-dorsalizing morphogenetic protein-1; antiandrogen, prostatic carcinoma: antiestrogen; antineoplaston; antisense oligonucleotides; aphidicolin glycinate; apoptosis gene modulators; apoptosis regulators; apurinic acid; ara-CDP-DL-PTSA, arginine deaminase; asulacrine; atamestane; atrimustine; axinastatin 1; axinastatin 2; axinastatin 3; azasetron; azatoxin; azatyrosine; baccatin III derivatives; balanol; batimastat, BCR/ABL antagonists; benzochlorins: benzoylstaursporine; beta lactam derivatives; beta-alethine; betaclamycin B; betulinic acid; bFGF inhibitor, bicalutamide; bisantrene; bisazindinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine: cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthrequinones; cycloplatam; cypemycin; cytarabine ocfosfate; cytolytic factor; cytostatin; dacliximab; decitabine; dehydrodidemnin B; deslorelin; dexifostamide; dexrazoxane; dexverapamil; diaziquone; didemnin B; didox; diethylnorspermine; dihydro-5-azacytidine; dihydrotaxol, 9-: dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane: fadrozole; fazarbine; fenretinido; filgrastim; frnasteride; flavopiridol; flezelastine; fluasterone; fludarabine; fluorodaunorunicin hydrochloride; torfenimex; formestane; fostriecin; fotemustine; gadolinium texaphyrin; gallium nitrate; galocitabine: ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists, interferons; interleukins; iobonguane; iododoxorubicin; ipomeanol, 4; trinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin: letrozole; leukemia inhibiting factor; leukocyte alpha interferon; leuprolide+estrogen+progesterone; leuprorelin; levamisole liarozole; linear polyamine analogue; lipophilicadisaccharide peptide; lipophilic platinum compounds; lissoclinamide-7: lobaplatin, lombricine: lometrexol: lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosplioryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin: nitric oxide modulator; nitroxide antioxidant; nitrullyn; O6-benzylguanine; octreotide; okicenone; oligonucleotides; onapristone; orldarisetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol: panomifene: parabactin; pazelliptine; pegaepergase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin: piritrexim; placetin A; placetin B; plasminogen activator inhibitor; platinum comprex; platinum compounds; platinum-triamine coil iplex; porfimer sodium; portiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purino mucleoside phosphorylast inhibitors; purpurins; pyrazoloacridine; pyridoxylated hemoglobin polyoxyethylene conjugate; raf antagonists; raltitrexed; ramosetron; ras farnesyl protein transferase inhibitors: ras inhibitor,: ras-GAP inhibitor, retalliptine demethylated; rhenium Re186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim, Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmonine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin: toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erytrocyte gene therapy; velaresol; venom, anti-venom, veramine; verdins; verteporfin; vinorelbine; vinxaltine; vitaxin; vorozole; zanoterone; zeniplatin; zilascorb; zinostatin stimalamer, immunostimulating drugs or therapeutic agents, their metabolites, salts and derivatives thereof.

53. The use of a pharmaceutical formulation as claimed in claims 50 to 52, wherein the pharmaceutical formulation is administered in combination with radiation treatment.

54. The use of a pharmaceutical formulation as claimed in claims 50 to 53, wherein the pharmaceutical formulation is administered in combination with surgery.

55. The use of a pharmaceutical formulation as claimed in claims 50 to 54, wherein the neoplasia is a precancerous lesion including syndromes represented by abnormal neoplastic and/or dysplastic, changes of tissue comprising precancerous growths in colonic, breast, renal, central nervous, gastric, or lung tissues, or conditions such as dysplastic nevus syndrome, a precursor to malignant melanoma of the skin, dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

56. The use of a pharmaceutical formulation as claimed in claims 50 to 55, wherein the neoplasia is prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas, Hematopoeitic cell cancers such as B cell leukaemia/lymphomas, myelomas, T-cell leukemias/lymphomas, small cell leukemias/lymphomas, null cell, sezary, monocytic, myolomonocytic and Hairy cell leukemias.

57. The use of a pharmaceutical formulation as claimed in claims 50 to 56, wherein the neoplasia is in the form of a tumour comprising an epidermoid and myeloid tumour, acute or chronic, nonsmall cell, squamous or solid.

58. The use of a pharmaceutical formulation as claimed in claims 50 to 57, wherein the composition is micronized.

59. The use of a pharmaceutical formulation as claimed in claims 50 to 58, wherein the pharmaceutical formulation has an enteric coating.

60. The use of a pharmaceutical formulation as claimed in claim 59, wherein the enteric coating is made of a polymer or copolymer.

61. The use of a pharmaceutical formulation as claimed in claim 60, wherein the polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

62. The use of a pharmaceutical formulation as claimed in claims 50 to 61, wherein, the pharmaceutical formulation is administered enterally, parenterally, topically, orally, sublingually, rectally, nasally or vaginally.

63. The use of a pharmaceutical formulation as claimed in claims 50 to 62, wherein the composition is formulated into liposomes or carbohydrate vehicles.

64. The use of a pharmaceutical formulation as claimed in claim 63, wherein the liposomes or carbohydrate vehicles are specifically targeted to tumours by covalently attaching a monoclonal antibody directed to a tumour-associated antigen.

65. The use of a pharmaceutical formulation as claimed in claims 50 to 64, wherein the pharmaceutical formulation is administered intermittently.

66. The use of a pharmaceutical formulation as claimed in claims 50 to 65, wherein the pharmaceutical formulation is a unit dose that comprises 5-500 mg of active ingredient consisting of at least one compound of the present invention.

67. The use of a pharmaceutical formulation as claimed in claims 50 to 66, wherein the pharmaceutical formulation is administered to a mammal.

68. The use of a pharmaceutical formulation as claimed in claim 67, wherein said mammal is a neonate and said administering is effected prior to delivery of said neonate and/or during delivery of said neonate.

69. The use of a pharmaceutical formulation as claimed in claims 50 to 68, wherein the composition acts as a prodrug.
